(19) United States
(12) Patent Application Publication
(10) Pub. No.: US 2015/0032032 A1
(43) Pub. Date: Jan. 29, 2015

(54) METHODS AND DEVICES FOR BIOMECHANICAL ASSESSMENT OF PELVIC FLOOR INCLUDING PERINEUM PRIOR TO CHILDBIRTH

(71) Applicants: Vladimir Egorov, Princeton, NJ (US); Vladimir Kalis, Plzen (CZ); Zdenek Rusavy, Plzen (CZ)

(72) Inventors: Vladimir Egorov, Princeton, NJ (US); Vladimir Kalis, Plzen (CZ); Zdenek Rusavy, Plzen (CZ)

(73) Assignee: ADVANCED TACTILE IMAGING INC., Trenton, NJ (US)

(21) Appl. No.: 14/494,480

(22) Filed: Sep. 23, 2014

Related U.S. Application Data
(63) Continuation-in-part of application No. 13/756,788, filed on Feb. 1, 2013, now Pat. No. 8,840,571.

(51) Int. Cl. A61B 5/00; A61B 5/11; A61B 5/107

(52) U.S. Cl. CPC A61B 5/4337; A61B 5/1076; A61B 5/4839; A61B 5/4362; A61B 5/11; A61B 5/7275
USPC 600/591

(57) ABSTRACT

The transvaginal tactile probe is configured to obtain a high resolution mapping of pressures and strains within the vagina of a pregnant woman prior to birth. The device provides real-time data visualization, analysis tools and information. This data may be used to assist with clinical decisions regarding selecting a preferred method of prevention of severe childbirth injury or altering delivery management, e.g. early induction at term, elective caesarean section in patients with a history of obstetric anal sphincter injury, water-birth, warm compresses to the perineum. The device is intended for use by medically trained personnel who counsels patients regarding risk of severe trauma at childbirth (such as urogynecologists and obstetricians) and regarding the effect of perineal massage and childbirth training device (such as physical therapists).

### CROSS-REFERENCE DATA

This application is a continuation-in-part of U.S. patent application Ser. No. 13/756,788 filed Feb. 1, 2013 entitled “Method and device for measuring tactile profile of vagina”, now U.S. Pat. No. 8,840,571, which claims the priority benefit of the U.S. Provisional Patent Application No. 61/617,555 filed Mar. 29, 2012 and in turn is a continuation-in-part of a U.S. patent application Ser. No. 13/439,165 filed Apr. 4,2012 entitled “METHODS FOR ASSESSMENT OF IMPROVEMENTS IN PELVIC ORGAN CONDITIONS AFTER AN INTERVENTIONAL PROCEDURE”, now U.S. Pat. No. 8,419,659, which in turn is a divisional of U.S. patent application Ser. No. 13/083,494 filed 8 Apr. 2011 entitled “Methods for assessment of pelvic organ conditions affecting the vagina”, now U.S. Pat. No. 8,187,208, which in turn is a continuation-in-part of U.S. patent application Ser. No. 12/874,583 filed 2 Sep. 2010 entitled “Methods for characterizing vaginal tissue elasticity”, now U.S. Pat. No. 8,052,622, which in turn claims a priority benefit from a U.S. Provisional Patent Application No. 61/239,087 filed 2 Sep. 2009 entitled “Methods of using a vaginal tactile imager for pelvic organ prolapse characterization, including that after a reconstructive surgery”, all of which are incorporated herein in their respective entireties by reference.

### BACKGROUND OF THE INVENTION

The present invention relates generally to probes and methods for detecting tissue elasticity. More particularly, the invention describes a transvaginal probe and methods for measuring stress and strain of tissues of and adjacent to vaginal canal prior to childbirth. The invention further describes selecting the preferred method of childbirth or assisting with childbirth based on such measurements.

Extensive deformations of the pelvic floor structures occur in the course of a childbirth using vaginal delivery. At the level of perineum, these deformations can be associated with various extent of trauma, which may even lead to an obstetric anal sphincter injury. Approximately 85% of women suffer some extent of perineal trauma during a vaginal delivery of a baby and in about 69% suturing or other surgical repair is required. The trauma during childbirth may lead to functional problems in the future. Perineal pain is reported by as much as 42% of women ten days after delivery and it prevails for more than one and a half year in about 10% of them. In addition, literature suggests that up to 58% of women complain of dypareunia as long as 3 months postpartum. Postpartum perineal trauma is a distressing event significantly contributing to overall postpartum morbidity and frustration of women after delivery. Since it is a world-wide problem concerning all women and women are increasingly aware of the detrimental effects of delivery on their pelvic floor and asking for the least traumatic delivery possible, it is important to devise strategies for reduction the incidence and the extent of such trauma. Therefore, new tools and methods are needed to improve counseling of these women and provide for the least traumatic experience during childbirth.

Perineal elasticity may play an important role in the etiology and pathophysiology of perineal trauma at childbirth. No device capable of reliable assessment of the perineal elasticity exists. A new device that could provide information on the elasticity of the perineum of a pregnant woman may be very useful for counseling the women regarding possible interventions to reduce perineal trauma or selecting a preferred mode of delivery. Techniques such as perineal massage or use of EPI-NO Childbirth Trainer may be advised and their effectiveness may be evaluated. Knowledge of perineal elasticity may therefore constitute an important factor in the decision making regarding labor induction/caesarean section for a large fetus in order to preserve perineal tissues and function. No such device exists so far and subsequently no study exists that would prove the hypothesis that perineal elasticity plays a key role in the pathophysiology of severe perineal trauma and OASIS exists.

There is a need therefore for a novel device to assess vaginal tissue elasticity with the overall objective of predicting tissue trauma depending on the size of the baby as well as for objective selecting of the preferred method of delivery and preparation of vaginal canal therefor.

### SUMMARY OF THE INVENTION

Accordingly, it is an object of the present invention to overcome these and other drawbacks of the prior art by providing a novel transvaginal probe configured to provide biomechanical profile of the vaginal canal prior to childbirth.

It is another object of the present invention to provide novel methods of biomechanical assessment of vaginal canal and surrounding tissues including perineum in order to determine the risk of trauma depending on the size of the baby, selected method of childbirth and other factors.

It is a further object of the present invention to provide novel methods of counseling pregnant women as to preferred methods of childbirth based on biomechanical profile of the vaginal canal and perineum prior to childbirth.

Described herein are various designs of the transvaginal tactile probe specifically configured for assessment of vaginal and perineum tissue elasticity prior to childbirth. The probe has a double-curved distal portion with a convex part housing the tactile sensors. This shape resembles that of a baby's head so as to facilitate the probe to simulate the movement of the baby's head through the vaginal canal.

Methods of the invention include determining of tactile profile of at least certain portions of the vaginal canal including that in the vicinity of the pelvic bone. A risk assessment may then be conducted to evaluate the likelihood of tissue damage and rupture during natural childbirth. The methods may also be used for predicting the benefit of and recommending the preferred interventional approach before and during delivery aimed at assisting the woman and reducing the probability of perineal and vaginal damage.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a size view of a procedure of a vaginal examination using the probe of the invention;

FIG. 2 shows exemplary transverse, sagittal and coronal cross-sections of a 3-D tactile image of vagina for a patient with known normal pelvic floor conditions examined with the transvaginal probe of the invention;

FIG. 3 is a flow chart illustrating steps of one method for biomechanical assessment of pelvic floor including perineum prior to childbirth;

FIG. 4 is a schematic diagram of the transvaginal probe with its internal components; and

FIG. 5 shows perspective and side views of a transvaginal probe of the invention.

### DETAILED DESCRIPTION OF THE PREFERRED EMBODIMENT OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

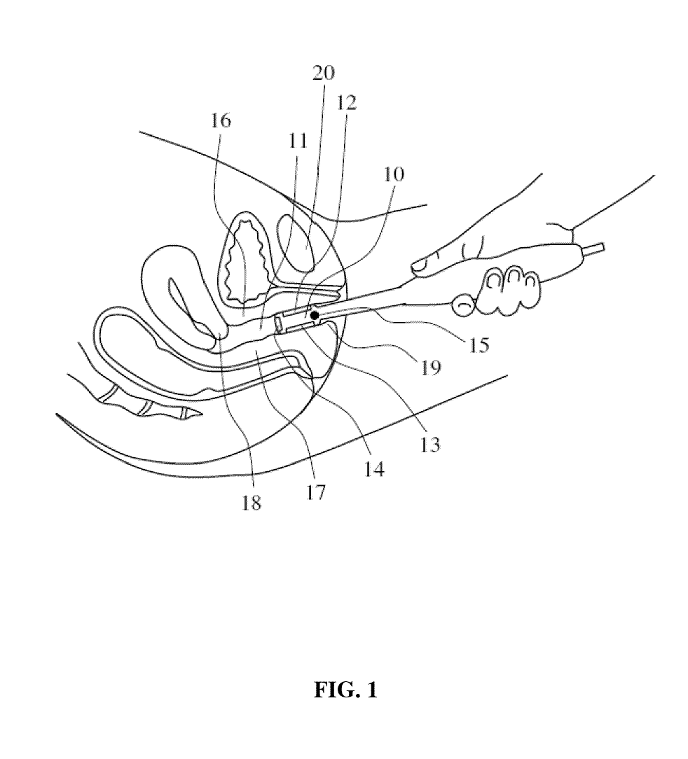

Referring now to FIG. 1, a general procedure of a vaginal examination is illustrated. The examination may be performed on patient in a standard position suitable for physical examination of the birth canal (vagina) in an obstetric office. During examination, the transvaginal probe 10 may be protected by a disposable elastic sheath covered with a lubricant. The transvaginal probe 10 may be placed into vagina 11 and used to deform the vaginal wall and surrounding pelvic floor muscles by applying a pressure load thereto. In embodiments, an exemplary examination technique includes sequential compressions of the posterior vaginal wall 17 by the probe 10 from the proximal to distal part of the vagina to collect the data along the posterior vaginal wall, allowing visualization of the posterior part of vagina in real time and calculation of an elasticity modulus profile along the examined posterior vaginal wall. Turning the probe and using the same technique, an operator may receive vaginal tactile images and elasticity modulus profiles along the examined anterior vaginal wall 16, as well as left and right sides of the vagina (not shown). Tactile imaging data coming from the transvaginal probe may be observed in three orthogonal projections representing examined parts of the vagina. A sliding motion along the vaginal canal of the probe 10 while exerting a load to the vaginal wall may also be used during the examination to provide a circumferential vaginal image.

Other examination techniques are also applicable for the purposes of the invention. For example, during the examination procedure, the transvaginal probe 10 may be moved to perineum 19 and a mechanical load may be applied to the perineum to assess its elasticity capability for 3-7 cm deformations in sagittal plane. The pelvic bone 20 may also be investigated to assess the distance between the stretched perineum and the pelvic bone as one the most critical parameters during the child delivery, which may be used to assess the likelihood of tissue trauma and rupture.

An average pressure applied to the vaginal wall may be in the range from about 10 kPa to about 20 kPa. The term “about” is used here and throughout this description to mean +/−30% variation of the cited parameter. The vaginal examination procedure may be performed once or may be repeated several times at one or more selected locations in vagina. In embodiments, additional passes of the probe 10 over one or more locations along the vaginal walls may be conducted with increasing levels of the pressure, for example up to about 40 kPa. Obtained data may be stored in a digital format allowing a review of one, two, or three orthogonal cross-sections selected for tactile image of vagina. Tactile images of vagina may be used for calculating elasticity modulus at specified locations or along selected lines and geometrical features. In embodiments, a spacing profile may for example be calculated as a set of distances between anterior and posterior along the vagina in at least two or more such locations.

The timing of such evaluation may be selected to be near the projected due date for the subject. In embodiments, such procedure may be performed once or repeated several times within the last 5-10 days prior to expected childbirth. This evaluation procedure may also be performed prior to childbirth in cases of premature birth or late birth. In other embodiments, this procedure may be first performed weeks and even 1-3 months prior to due date and optionally repeated closer to the projected due date.

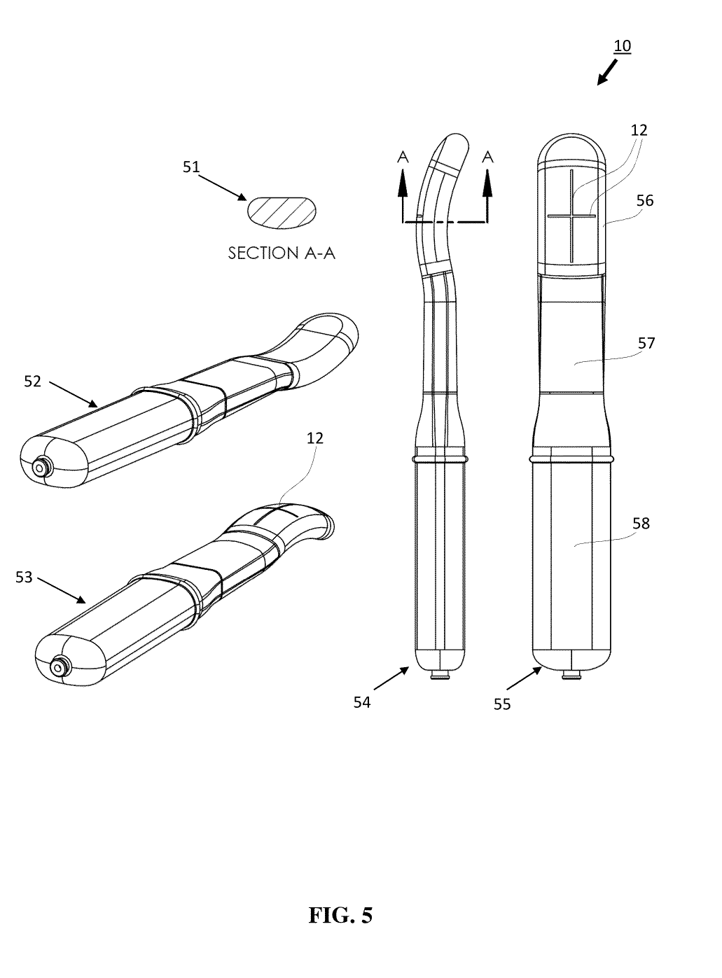

The transvaginal probe 10 may include one or multiple pressure sensors forming one or multiple pressure sensor arrays configured for atraumatic contacting vaginal walls and cervix. As shown in FIG. 1, sensor arrays 12 and 13 may be configured for a contact with opposing vaginal walls, e.g. anterior wall 16 and posterior wall 17, as well as a left side and a right side of vagina 11. Pressure sensor array 14 may also be configured for a contact with cervix 18. The pressure sensor arrays of the probe 10 may be assembled as two-dimensional sensor arrays on all or part of the exterior surface of the probe 10 and adapted for contacting the vaginal tissue. The pressure-sensitive surface of the probe 10 configured for contacting the vaginal wall may have a rounded shape with an exemplary radius of curvature of about 10 mm to about 30 mm. In embodiments, this radius of curvature may be about 15 mm. The pressure-sensitive surface of the pressure sensor array 14 may be double-curved in the shape of ellipsoid with curvature radius from about 20 mm to about 55 mm as may be seen in FIG. 5 the external convex area of this ellipsoid may be used to house the tactile sensors of the probe. The probe may further include a motion tracking sensor 15 configured to record at least one or more of the three coordinates (X, Y, Z) and/or three angles (Elevation, Rotation, Azimuth) of the transvaginal probe 10. Recording of these coordinates and angles allows calculating coordinates of all pressure sensors of the probe 10 in a coordinate system which may be tied to the pelvic floor bone framework. An electronic unit for data acquisition may be provided and configured to record the pressure array readings and the motion sensor readings, so that in combination, the sensor coordinates and sensor pressure data may be recorded at the same time and paired together for detecting stress-strain distribution and producing a tactile profile of the tissues such as for example a 3-D tactile image.

The pressure pattern on the surface of the vagina wall while deformed by the probe 10 reveals not only elasticity conditions of vaginal wall itself, but an elasticity distribution of underlying structures: higher applied pressure reveals deeper structures surrounding vagina. The pressure patterns on the surface of vaginal walls together with tissue displacement caused by deformation from the probe 10 may be considered as documentation of the current elasticity state of the vaginal walls and surrounding support structures. This information can be used in prediction of the extent of safe deformations of the birth (vaginal) canal.

Acquired pressure patterns form together a tactile profile of the vaginal and perineum tissues, which may be represented as a 2-D or a 3-D tactile image. The 3-D tactile image of the vagina may be composed using a spatial scalar mapping of the pressure patterns acquired at the vaginal wall under deformation. The spatial mapping technique may involve acquisition of probe motion tracking data, which may be then transformed into spatial coordinates of each pressure sensor at the contact surface of the vaginal wall where the pressure signals were acquired during in the course of vaginal wall deformation.

The pressure (tactile) sensors may be configured to supply stress data and the motion tracking sensor may be configured to supply strain data. These stress and strain data allows assessment of the vaginal tissue elasticity, predicting vaginal canal possible deformations during delivery and predicting possible tissue damages around vaginal canal during the delivery.

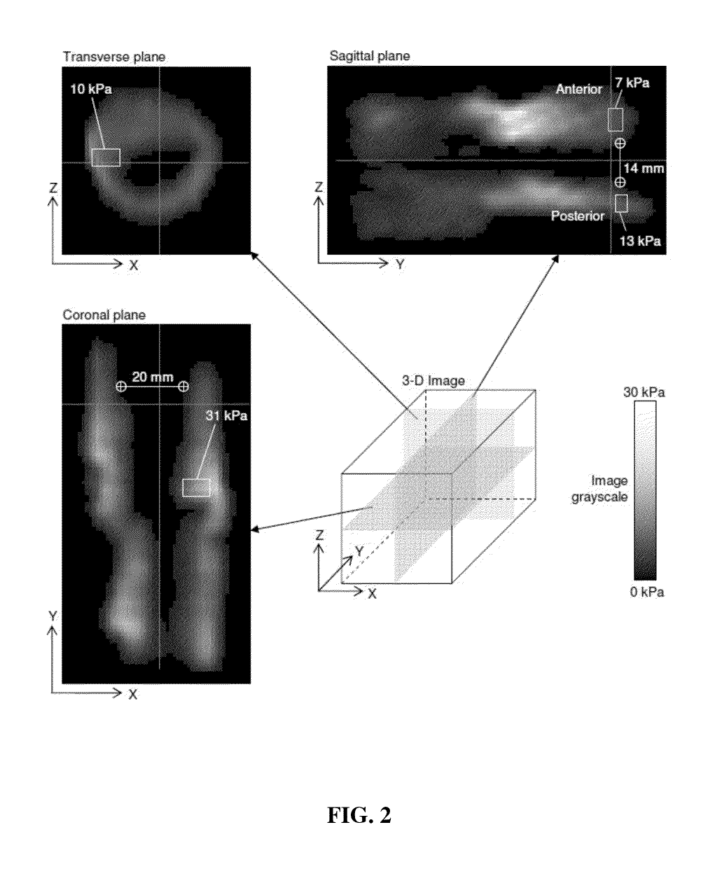

FIG. 2 shows an example of transverse, sagittal and coronal cross-sections of a 3-D tactile image of a normal vagina.

One practical way to observe a 3-D tactile image of vaginal canal may be to represent it by three orthogonal cross-sections of the vagina as shown in FIG. 2. The primary regions of interest may be the anterior and posterior parts of the vagina, which are represented on the sigittal cross-section (see Sigittal plane in FIG. 2). Circumferential vaginal structures may be better observed in the transverse cross-section (see Transverse plane in FIG. 2). Left and right side support structures may be visualized in coronal cross-section (see Coronal plane in FIG. 2). Tissue elasticity measures such as Young's modulus (E) may be calculated using a number of possible approaches, including:
(a) comparing spatial gradients in 3-D tactile images of the vagina with the spatial gradients in 3-D tactile images for tissue models of the vagina. Such models may be prequalified to have known distribution of Young's moduli,
(b) analyzing load curve for the probe during vaginal wall deformation applying a preexisting tissue deformation model, or
(c) using mechanical inverse problem solution applied to 3-D tactile image.

All three approaches require selection of a volume for averaging pressure gradients around the specified location where the tissue elasticity is to be calculated. Typical volume appropriate for that purpose may be in the range of about 50-100 mm³. The first calculating method from those listed above was used in exemplary results for tissue elasticity presented below.

Exemplary transverse, sagittal and coronal cross-sections of a 3-D tactile image of vagina in FIG. 2 are for a subject with known normal pelvic floor conditions examined with the transvaginal probe. The tactile image of the vagina is presented in the units of pressure (kPa) according to specified grayscale map. 3-D tactile images of the vagina may have also color-scale map, such as the jet map. In embodiments, an operator using touchscreen capabilities of software interface may select geometrical features inside shown cross-sections to be measured, displayed and recorded in a computer-generated examination report. Selected geometrical features may include spacing between anterior and posterior walls, between left and right vaginal walls, distance between any two selected points or size of the specified zone. The operator may also select specific sites for calculation of tissue elasticity features, such as elastic modules, e.g. Young's modulus. Characteristic anatomical measures may be placed in the images and tissue elasticity values (Young's moduli) may be calculated for different sites. Comparison of different locations demonstrates the distribution of tissue elasticity. The sigittal and transverse tactile image cross-sections in FIG. 2 demonstrate strong anterior and posterior vaginal support with anterior-posterior spacing at distal part of about 14 mm. Young's modulus (E) was calculated for areas specified by a rectangular: E=7 kPa at distal anterior and E=13 kPa at distal posterior sections of vagina respectively. Right side of vaginal distal part demonstrated E=10 kPa (see Transverse plane in FIG. 2).

Calculated tissue elasticity and geometrical measures may be projected on the respective cross-section of the 3-D tactile image to assist in visual interpretation of the examination results and comparison with reference data. The reference data may include prior measurements for the same patient, expected values of tissue deformation and predicting tissue damages during the delivery.

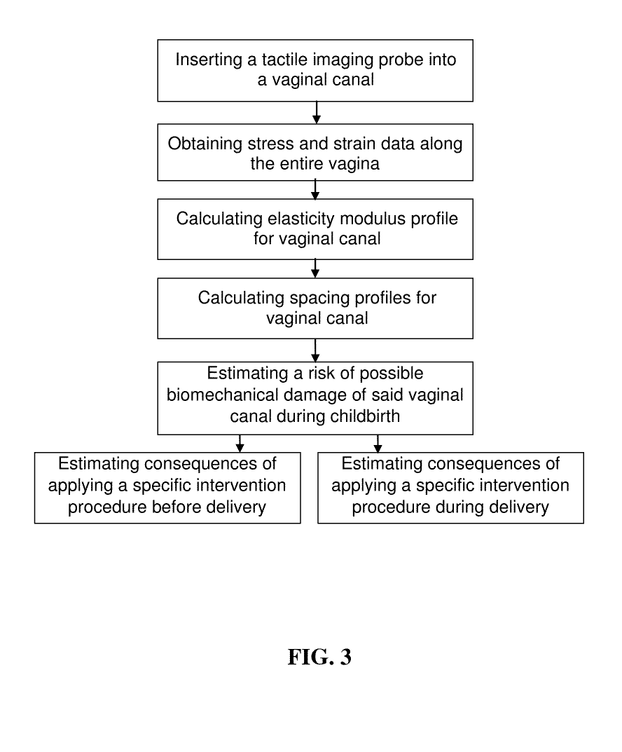

Referring to FIG. 3, a method for biomechanical assessment of pelvic floor including perineum prior to childbirth is depicted as a block-diagram. The method may include the steps of:
(a) inserting a transvaginal probe 10 into vagina along a vaginal canal to separate apart two opposing vaginal walls thereof, whereby causing vaginal tissue deformation away from the vaginal canal,
(b) obtaining stress and strain data along a part or the entire vaginal canal including perineum and pelvic bone areas using the transvaginal probe, the stress and strain data in this case are indicative of the deformation applied to the vaginal tissue;
(c) calculating from the stress and strain data one or more elasticity modulus profiles defined by at least two or more paired and opposite locations in the vagina. Such elasticity modulus profile may be an anterior elasticity modulus profile, a posterior elasticity modulus profile, a left side elasticity modulus profile or a right side elasticity modulus. This step may be expanded by calculating from the stress and strain data at least one of an anterior-posterior spacing profile or a left-right spacing profile. The anterior-posterior spacing profile may be defined by at least two distances between the anterior vaginal wall and the posterior vaginal wall. The left-right spacing profile may be defined by at least two distances between the left side and the right side of the vagina;
(d) estimating a risk of biomechanical damage of said vaginal canal during childbirth. This may be done using additional information such as baby size and weight and in particular using the size of the baby's head. It may further be done using a clinical database with known delivery outcomes including severity and locations of tissue damage for a variety of subjects with known elasticity profiles of vaginal canal and perineum acquired before childbirth; and
(e) optionally estimating consequences of application of a specific intervention procedures before or during delivery.

Step (e) is optional and may or may not be needed depending on the clinical circumstances. Step (e) may be implemented using a clinical database with records of known applied intervention procedures and delivery outcomes (tissue damages, their severity and locations) for a variety of elasticity modulus profiles for vaginal canal acquired before the delivery.

Above listed step (b) may further include recording of a transitional stress and strain data by simulating trajectory of a baby's head during the delivery and while inside the vagina. This may be accomplished by respective movement of the probe 10 inside the vagina simulating the movement of the baby's head causing the transvaginal probe to further deform the vaginal tissue, whereby facilitating a comprehensive characterization of biomechanical conditions of the vaginal canal during birth.

Step (c) may further include determining of tissue elasticity along and across of the perineum.

Step (d) may further include determining the baby size and in particular of the head diameter before the delivery. This may be accomplished by a variety of known techniques including ultrasound evaluations and predictions of the weight and size of the baby and the size of the baby's head.

Step (e) may further include selection of the most suitable intervention procedure prior or during childbirth for the purposes of minimizing the risk of pelvic floor tissue trauma or damage. Examples of such procedures may include steps aimed at increasing vaginal or perineal elasticity such as antenatal perineal massage, Epi-no® birth training device, perineal hyaluronidase (HAase) injection, induction of labor at term, or an indication for elective lower segment caesarean section (LSCS).

Step (e) may further include selection of an intervention procedure most suitable for specific vaginal canal, such as for example a manual perineal protection, episiotomy, Ritgen maneuver, warm packs on perineum during labor, perineal massage during labor, maternal birth position, various pushing methods during the second stage, use of obstetric gel, operative delivery with forceps vs. vacuum-extraction, water birth, epidural analgesia, etc.

The following is a general description of various known intervention techniques which may be selected as the most suitable according to the methods of the invention after assessment of vaginal and perineal tissue elasticity:

### I. Interventions Used Before Delivery

Antenatal perineal massage—includes massaging the perineum in a defined way several times a day in order to increase its elasticity. It is proven to be effective in reduction of perineal lacerations and rate of episiotomy. In nulliparas, perineal massage during the weeks before giving birth protected against perineal trauma. Such massage may be performed in the last several weeks before delivery to reduce trauma.

The probe 10 and the methods of the invention may be used to examine the subject regarding her perineal elasticity and need for this procedure. Furthermore, it may be used to track the progress of the subject, i.e. whether the massage is effective.

A birth training device, such as Epi-no® or a similar device used to increase perineal elasticity—is a device intended for stretching of the pelvic floor muscles and perineum. Published studies have demonstrated a significant reduction in the rate of episiotomies in the group of women who participated in the birth training program with Epi-no® in the weeks prior to childbirth. Another benefit is a strong decrease in the rate of perineal tears: it was twice as low in patients using the Epi-no® birth training device as compared with control.

The probe 10 may be used to assess the need for such training and the potential effectiveness of the training prior to the delivery.

Perineal hyaluronidase (HAase) injection—this was widely used to reduce the occurrence of perineal trauma, pain and need for episiotomy in the 1950s to 1960s. Published data from three trials involving 373 women suggested that perineal HAase injection during second stage of labor had a lower incidence of perineal trauma compared with no intervention, but had no clear effect on in the incidence of episiotomy and first and second degree perineal lacerations.

The probe and methods of the invention may be used to assess the need for perineal HAase injection prior to the delivery as well as its effectiveness in the first stage of labor.

Induction of labor at term—this may be used before its spontaneous onset of labor in women at increased risk of perineal laceration. This procedure is already done in indicated cases, i.e. high-risk pregnancy, diabetes, macrosomic fetus, a parturient less than 150 cm tall, etc.

The probe and methods of the invention may be used to assess the elasticity of perineum as another indication for this procedure, i.e. perineum with high values of elasticity module may be viewed as being at increased risk of laceration.

Such high value threshold for this and other interventions of the present disclosure may be defined as exceeding 15 kPa, 20 kPa, 30 kPa, 40 kPa, 50 kPa, 70 kPa, 100 kPa or any value inbetween when defining Young's modulus—particularly in the sections of the vaginal canal adjacent to the perineum. Elasticity modulus profile may be analyzed together with the spacing profile of the vaginal canal. Specifically, it is important to assess spacing between the pubic bone and perineum at specified value of allied stress, e.g. about 30 kPa pressure on average being applied to perineum. Additional considerations may be given to the size of the vaginal canal with larger canals allowing for higher values of elasticity module thresholds to still avoid injury.

Elective lower segment caesarean section (LSCS)—Performing a LSCS on a pre-planned date may be done in order to avoid vaginal delivery in women with extreme risk of pelvic floor trauma or obstetric anal sphincter injury (OASI). This procedure is already indicated in certain cases, i.e. subjects with known OASI or some form of anal incontinence (in order to prevent deterioration of incontinence/repetition of severe trauma), subjects after pelvic floor reconstructive surgery or anti-incontinence procedures, subjects in whom a cephalopelvic disproportion is expected (small stature, macrosomic fetus), women with very high estimated fetal weight (4,500 g, or 4,000 g in a diabetic), etc.

The methods of elasticity examination of the invention may be used to add another indication, i.e. a scarified perineum may be determined to be at increased risk of OASI.

### II. Interventions Used During the Delivery

Manual perineal protection—protection/support to the perineum may be provided in the final stages of the second stage of labor. There are many different methods/maneuvers that may be used for this purpose. Although the benefit is expected, it is yet to be demonstrated in large published studies. Clear benefit from implementation of perineal protection intervention programs was recently demonstrated in some countries.

The new device and methods described herein may add information regarding the necessity of this procedure. This may be especially important for women who want to deliver in a way that makes manual perineal protection impossible to perform (water-birth, standing position, squatting position etc.). The device of the invention may help in developing data supporting proper counseling the women regarding their risk of severe lacerations should they decide to deliver in such way.

Episiotomy—is a surgical enlargement of the vaginal orifice in order to facilitate or expedite delivery. There are several types of this procedure known to be used in clinical practice, with median, mediolateral and lateral being the most common. One of the most frequent indications for this procedure is inelastic perineum, which is assessed subjectively after manual examination.

The device and methods of the invention may be used to add information regarding the necessity of this procedure. It may allow for an objective assessment of the perineal elasticity and define the inelastic perineum, a condition that can benefit from elective episiotomy in order to avoid a severe perineal tear. It may also be used when needing to expedite delivery in case of a fetal distress—the knowledge of perineal elasticity could help in choosing a proper procedure that would lead to a fast delivery of the fetus with minimal trauma to the mother, such as deciding between Ritgen maneuver and no episiotomy vs. episiotomy.

Ritgen maneuver—defined as facilitating extension of the fetal head by applying pressure to the posterior perineum during labor in order to reduce the extent of perineal trauma or expedite delivery. This may be suitable for women with elastic perinea. The benefit of the procedure was first demonstrated long time ago. The perineal elasticity is an important factor to consider when selecting this intervention.

The new devices and methods of the invention may add information regarding the possible benefit of the procedure. Objective assessment of perineal elasticity and detection of “elastic” perineum may be instrumental to indicate when the modified Ritgen maneuver could be performed safely to expedite delivery with reduced trauma (vs. cutting an episiotomy).

Warm packs on perineum during labor—this may be helpful in order to increase the blood flow and increase the elasticity. A significant effect of warm compresses on reduction of third- and fourth-degree tears was shown in the literature.

The device and methods of the invention may be used to assess the potential of effectiveness of the intervention. When done prenatally, it may be used to assess the potential of the perineum to increase its elasticity when warmed up.

Perineal massage during labor—massaging perineum during labor in order to increase its elasticity was shown to have a significant effect towards favoring massage versus hands off to reduce third- and fourth-degree tears.

The present invention may help to assess the effect of this intervention. When done prenatally, elasticity evaluation may assess the potential of the perineum to increase its elasticity when massaged during the labor.

Maternal birth position—positioning of the mother in the second stage of labor to facilitate delivery and/or reduce perineal trauma may be done using several positions: supine, semi-recumbent, lithotomy, lateral and upright positions—standing, sitting, squatting and kneeling. Birthing chair or stool may be used. The extent of perineal trauma in certain positions where manual perineal protection is impossible may depend on perineal elasticity.

No consensus exists regarding negative impact or benefits of upright positions on the mother. Reduction of episiotomy, slight increase in the second degree tears and more frequent blood loss over 500 ml have been described. The mother's position during the second stage may have little influence on perineal trauma. Different positions might involve different risks of the third degree tear.

As the mother's preference needs to be respected with regard to the way they want to deliver their babies, the new device and methods may be used to help in counseling them and possibly estimate whether their perineum is at increased risk of laceration due to its low elasticity.

Pushing methods during the second stage of labor—cooperation between mother and obstetrician/midwife at the time of expulsion of the fetal head is critical. Slowing the passage of the fetal head through the perineal structures may help in avoiding an injury. The method may be used to slow down the expulsion of the fetal head, for example to deliver between contractions so as to reduce the risk of perineal trauma compared to delivering during contraction while pushing. If perineum is found to be sufficiently elastic using the devices and methods of the present invention, the woman may be counseled to not have to slow down the speed of delivery during these final most painful moments.

Use of obstetric gel—this is used to decrease the frictional forces during the movement of the baby through the birth canal. A significantly higher proportion of intact perinea was reported in the group using the gel compared to controls. In case of perineal inelasticity as may be defined using the methods of the present invention, obstetric gel may be used to facilitate the delivery, increase the rate of intact perineum during labor and shorten the final painful moments of vaginal delivery. The gel may be applied regularly during labor and after each contraction during the second stage to increase the integrity of the perineum.

Operative delivery—with forceps or vacuum-extraction of the head—an instrument (forceps or vacuum extractor) may be used to expedite the delivery of the fetus during the second stage of labor. Vacuum extraction and spontaneous birth was shown to cause less anal sphincter trauma compared with forceps delivery. If perineum is found inelastic using the present invention, the avoidance of instrumental delivery (or at least forceps) may reduce the risk of severe perineal trauma.

Water birth—the mother is immersed into water (having a bath) and baby is delivered under water. Studies have mixed reports on the extent of perineal injury using water bath delivery. Objective elasticity assessment tools are needed to define the risk of injury—if perineum is found to have high values of elasticity modulus as described above, the counseling may take place regarding all preventive steps (manual perineal protection in particular) with easy and sufficient access to the woman's perineum is possible.

Epidural analgesia—use of epidural analgesia may reduce the pain during delivery. It is also occasionally considered a risk factor for perineal trauma. If the women does not feel the perineum stretch, she can tear it more easily. However, no consensus exists regarding this matter.

If perineum is found to have high elasticity modulus described above in greater detail, a detailed counseling regarding risks and benefits of epidural analgesia with respect perineal integrity preservation is necessary.

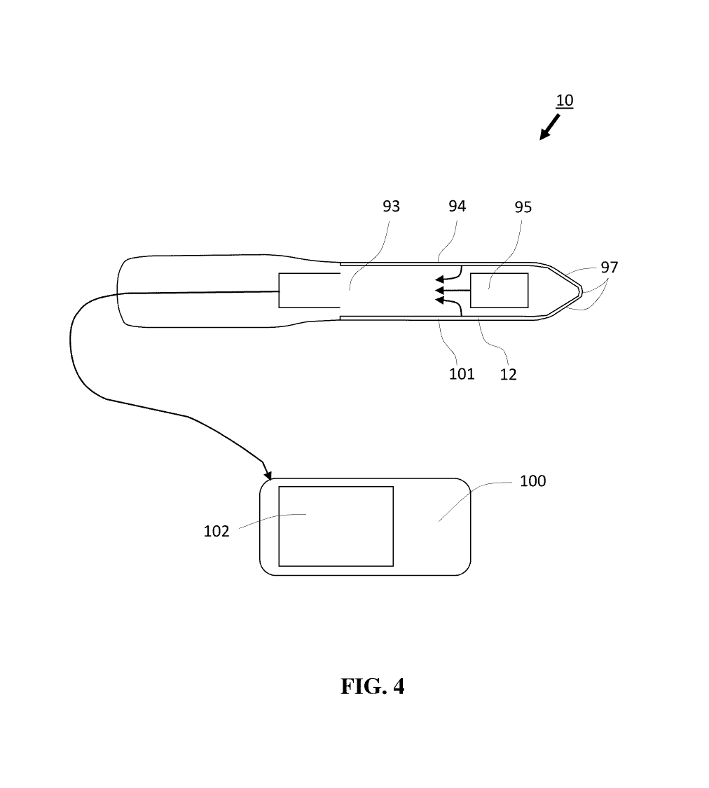

FIG. 4 is a general schematic diagram of a transvaginal probe 10 with its internal components. The probe 10 comprises a tactile sensor array 12 configured to record tactile signals from vaginal walls at applied levels of tissue deformation, a motion tracking sensor 95, and a data acquisition electronics 93 communicating with the external data processor 100. The motion tracking sensor may provide one-, two-, three-, four-, five-, or six-degree of freedom motion tracking data which include three coordinates (x,y,z) and three angles (azimuth, elevation, rotation). Furthermore, the data processor 100 may include a display 102 to present examination data with vaginal tactile profiles. The probe 10 may have flat or curved parts 94 and 101 to contact the vaginal walls. Tactile sensors of the array 12 may be located to cover some or all portions 94, 97, and 101 of the probe 10. Data transfer between the probe 10 and data processor 100 may be provided by a wired or wireless connection.

The controller may be configured to calculate projected biomechanical damage of the vaginal canal during childbirth, to estimate consequences (such as numerical risk of damage or tissue tear) of application specific intervention procedures before or during delivery of the baby.

FIG. 5. Shows various perspective views of the transvaginal probe 10 configured for the purposes of the invention. The probe 10 comprises a tactile sensor array 12 configured to record tactile signals from all parts of vaginal canal. The probe 10 has a distal portion 56 with the tactile sensors 12, which are configured as a cross with two orthogonal line sensor arrays. The tactile sensors may have a grip (step) of 2-5 mm along the line. The probe 10 also may have a shaft 57 and a handle 58. The probe's distal portion 56 may be shaped to resemble a part of a baby's head with the external convex portion housing the tactile sensors of the array 12.

The herein described subject matter sometimes illustrates different components or elements contained within, or connected with, different other components or elements. It is to be understood that such depicted architectures are merely examples, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

Although the invention herein has been described with respect to particular embodiments, it is understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A method for a biomechanical assessment of pelvic floor including perineum prior to childbirth comprising the following steps:
a. inserting a transvaginal probe into vagina along a vaginal canal to separate apart two opposing vaginal walls thereof, whereby causing vaginal tissue deformation,
b. obtaining stress and strain data along the vaginal canal using said transvaginal probe,
c. calculating based on said stress and strain data at least one elasticity modulus profile, and
d. based on said calculated elasticity modulus profile, estimating a risk of biomechanical damage of said vaginal canal during childbirth.

2. The method as in claim 1, wherein said at least elasticity modulus profile is selected from a group consisting of an anterior elasticity modulus profile, a posterior elasticity modulus profile, a left side elasticity modulus profile, and a right side elasticity modulus.

3. The method as in claim 1, wherein said step (c) further comprising a step of calculating from said stress and strain data at least one of an anterior-posterior spacing profile or a left-right spacing profile, said anterior-posterior spacing profile is defined by at least two distances between said anterior vaginal wall and said posterior vaginal wall, said left-right spacing profile is defined by at least two distances between said left side and said right side of said vagina.

4. The method as in claim 1, wherein said step (d) further comprising a step of estimating consequences of applying a specific intervention procedure before delivery.

5. The method as in claim 4, wherein said specific intervention procedure before delivery is selected from a group consisting of an antenatal perineal massage, use of a birth training device, use of a device to improve perineal elasticity, Perineal hyaluronidase injection, induction of labor at term, and elective caesarian section.

6. The method as in claim 1, wherein said step (d) further comprising a step of estimating consequences of applying a specific intervention procedure during delivery.

7. The method as in claim 6, wherein said specific intervention procedure during delivery is selected from a group consisting of a manual perineal protection, episiotomy, Ritgen maneuver, warm packs on perineum during labor, perineal massage during labor, maternal birth position, pushing methods during the second stage of labor, use of obstetric gel, operative delivery—with forceps or vacuum-extraction of the head, water birth, and epidural analgesia.

8. The method as in claim 1, wherein said step (b) further comprising a step of using said transvaginal probe to deform said vaginal canal to simulate movement of a baby's head therethrough while recording of transitional stress and strain data, whereby facilitating a comprehensive characterization of biomechanical conditions of said vaginal canal during childbirth.

9. The method as in claim 1, wherein said step (c) further comprising a step of determining tissue elasticity along and across of said perineum.

10. The method as in claim 1, wherein said step (d) further including determining of baby's head diameter.

11. A method of providing a recommendation for preferred method of childbirth, the method comprising the following steps:
a. obtaining a biomechanical assessment of pelvic floor including perineum prior to childbirth;
b. obtaining an assessment of fetus size prior to childbirth, and
c. based on said biomechanical assessment and said fetus size recommending a preferred method of childbirth.

12. A device for biomechanical assessment of pelvic floor including perineum comprising:
a. a transvaginal probe with a plurality of tactile sensors forming together a tactile array configured for acquisition of stress data, said plurality of tactile sensors are located over at least a portion of a double-curved distal portion of said probe shaped to resemble a baby's head;
b. a motion tracking sensor configured for acquisition of vaginal tissue strain data; and
c. a controller comprising a data processor configured for recording said stress and strain data, said data processor is further configured for determining said tactile profile of vaginal canal based on said stress and stress data.

13. The transvaginal probe as in claim 12, wherein said tactile sensors are configured in two orthogonal lines.

14. The transvaginal probe as in claim 8, wherein said controller is further configured to calculate a risk of biomechanical damage of said vaginal canal during childbirth.

15. The transvaginal probe as in claim 12, wherein said distal double-curved portion has an exterior convex area housing said tactile array.
